(19) United States
(12) Patent Application Publication
(10) Pub. No.: US 2016/0015939 A1
(43) Pub. Date: Jan. 21, 2016

(54) ABSORBENT AND ANTIMICROBIAL CUFF FOR CATHETER PLACEMENT

(71) Applicant: James T. Rawls, Folly Beach, SC (US)

(72) Inventor: James T. Rawls, Folly Beach, SC (US)

(21) Appl. No.: 14/804,594

(22) Filed: Jul. 21, 2015

Related U.S. Application Data
(60) Provisional application No. 62/026,983, filed on Jul. 21, 2014.

(51) Int. Cl. A61M 25/02

(52) U.S. Cl. CPC A61M 25/02; A61M 2025/0266; A61M 2025/0246; A61M 2025/026; A61M 2025/0253; A61F 2013/15048

(57) ABSTRACT

An cuff is described that facilitates the attachment of the catheter to a patient's skin for creating a secure attachment between the catheter and the patient's skin. The cuff that facilitates the attachment of the catheter to the patient's skin can be an absorbent and antimicrobial cuff. In some embodiments, the cuff can be utilized with a skin strip and/or a cover strip.

### CROSS REFERENCE TO RELATED APPLICATIONS

The present application claims filing benefit of U.S. Provisional Patent Application Ser. No. 62/026,983 having a filing date of Jul. 21, 2014, which is incorporated herein by reference in its entirety.

### BACKGROUND

Various types of catheters are well known in the medical field and are used throughout the world. For example, intravenous catheters are used for a wide variety of applications, including hydration and administration of intravenous fluids, medications, feeding, and transfusions. A typical catheter system utilizes an insertion device such as a needle or trocar for penetrating the skin and any other subdermal structures, e.g., a vein, and a catheter, the end portion of which remains in the patient following removal of the insertion device. Generally, during use the catheter is connected to medical tubing via a luer or other type of tubing connector.

Once placement in the patient has been confirmed, the external portion of the catheter is secured to the patient's skin with an adhesive tape until the catheter is removed by design, accidentally dislodged or damaged, or the tissue blows. The latter term (“blows”) is a common term for extravasation of the intravenous fluids and/or blood into the space outside of the targeted tissue, i.e., when the seal between the tissue and the catheter becomes leaky and fluid being administered thorough the catheter goes partly into other areas, e.g., external to the vein.

Unfortunately, the tape that is used to secure a catheter is impermeable and nonabsorbent, which leads to fluid accumulation under the tape and increases the likelihood of bacterial infection both at the catheter insertion site and in the local area where the skin is tightly covered by the tape. In addition, the area of skin that is covered by the tape is quite large, increasing discomfort for the patient.

What is needed in the art are devices and methods for securing a catheter following placement. For instance, a catheter securement device that is absorbable and/or antimicrobial would be of great benefit.

### SUMMARY

According to one embodiment, disclosed is a catheter stabilization device that includes a cuff that is configured to encircle or otherwise adhere to a portion, e.g., a length of a catheter. The cuff is configured to be directly or indirectly attachable to skin. For example, the cuff can include extensions that can be used to directly attach the cuff to skin. In one embodiment, the cuff can be absorbent and/or may also include an antimicrobial agent.

In one embodiment, the device can also include a cover strip that on a first side includes first, second and third portions. More specifically, the second portion is between the first and third portions along the length of the cover strip. The first and third portions can include an adhesive capable of temporarily attaching the cover strip to skin and thereby holding the cuff against the skin. The second portion can also be configured to be attachable to the cuff. In another embodiment, the first and third portions of the cover strip can be attachable to a skin strip. The skin strip can include an adhesive to attach to a wearer's skin on a first side and can be attachable to the cover strip at other portions of the second side. In this embodiment, the cuff can thus be ‘sandwiched’ between the skin strip and the cover strip and secured to a patient's skin. In one embodiment, the skin strip and/or the cover strip can also adhere to the cuff to more securely hold the cuff between the two strips.

Also disclosed is a method for stabilizing a catheter. For instance, the method can include inserting a catheter in a subject and then directly or indirectly attaching a cuff to the subject, the cuff being attached, e.g., encircling, the catheter on a portion, e.g., along a length, of the catheter. For instance, the cuff can be directly attached to the subject via adhesive of the cuff. In one embodiment, the cuff can be an absorbent and/or antimicrobial cuff. In one embodiment the cuff can be attached to the subject by use of a cover strip that can hold the cuff against the skin by adhesive portions of the cover strip. Alternatively, the cuff can be indirectly adhered to the subject by use of a skin strip that is attached on one side to the patient, for instance by an adhesive, and that can be attached to the cuff and/or the cover strip on a second side.

### BRIEF DESCRIPTION OF THE FIGURES

Various other objects, features and attendant advantages of the present invention will become fully appreciated when considered in conjunction with the accompanying drawings, in which like reference characters designate the same or similar parts throughout the several views, and wherein:

FIG. 1A is a top view of an insertion device attached to a catheter that includes a stabilization cuff.

FIG. 1B is a cross sectional view of a stabilization cuff.

FIG. 1C is a top view of a stabilization cuff including extensions for attachment to skin.

FIG. 2A is a top view of a skin strip that can attach to a patient's skin.

FIG. 2B is a top view of another embodiment of a skin strip that can attach to a patient's skin.

FIG. 2C is a top view of another embodiment of a skin strip that can attach to a patient's skin.

FIG. 3A is a top view of a cover strip that can be attached to a patient's skin and to a stabilization cuff.

FIG. 3B is a top view of another embodiment of a cover strip that can be attached to a skin strip and to a stabilization cuff.

FIG. 4 is a top view of an assembly at a catheter insertion site and illustrates an assembled securement device including a skin strip, cuff and cover strip.

FIG. 5 is a top view of an assembly at a catheter insertion site and illustrates a catheter held with two cuffs on a securement device and a 180° turn in the catheter.

### DETAILED DESCRIPTION

Objects and advantages of the present invention will become obvious and it is intended that these objects and advantages are within the scope of the present invention. To the accomplishment of the objects and advantages, this invention may be embodied in the form illustrated in the accompanying drawings, attention being called to the fact, however, that the drawings are illustrative only, and that changes may be made in the specific construction illustrated and described within the scope of this application, such changes being encompassed herein. Thus, it is to be understood that the invention is not limited in its application to the details of construction or to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

The present invention relates generally to catheter stabilization devices and more specifically it relates to an absorbent and/or antimicrobial cuff that facilitates the attachment of a catheter to a patient's skin. For example, in one embodiment the absorbent, antimicrobial cuff can be directly adhered to a patient's skin. In another embodiment, a catheter stabilization device can include an absorbent and/or antimicrobial cuff and a cover strip that can adhere the cuff to a patient's skin. In yet another embodiment, a catheter stabilization device can include an absorbent and/or antimicrobial cuff, a skin strip, and a cover strip.

One object of the present disclosure is to provide a stabilization cuff that facilitates the attachment of a catheter to a patient's skin for creating a secure and reliable attachment between the catheter and the patient's skin so that no occlusive plastic membrane dressing is required.

Another object is to provide a stabilization cuff that facilitates the attachment of the catheter to the patient's skin that decreases the risk of catheter dislodgement.

Another object is to provide an absorbent and/or antimicrobial cuff that facilitates the attachment of the catheter to the patient's skin that can decrease the risk of bacterial growth at the insertion site.

Another object is to provide an absorbent, antimicrobial cuff that facilitates the attachment of the catheter to the patient's skin that increases patient comfort.

Turning now descriptively to the drawings, in which similar reference characters denote similar elements throughout the several views, the figures illustrate a stabilization cuff 10, an adhesive skin strip 20, and a cover strip 30.

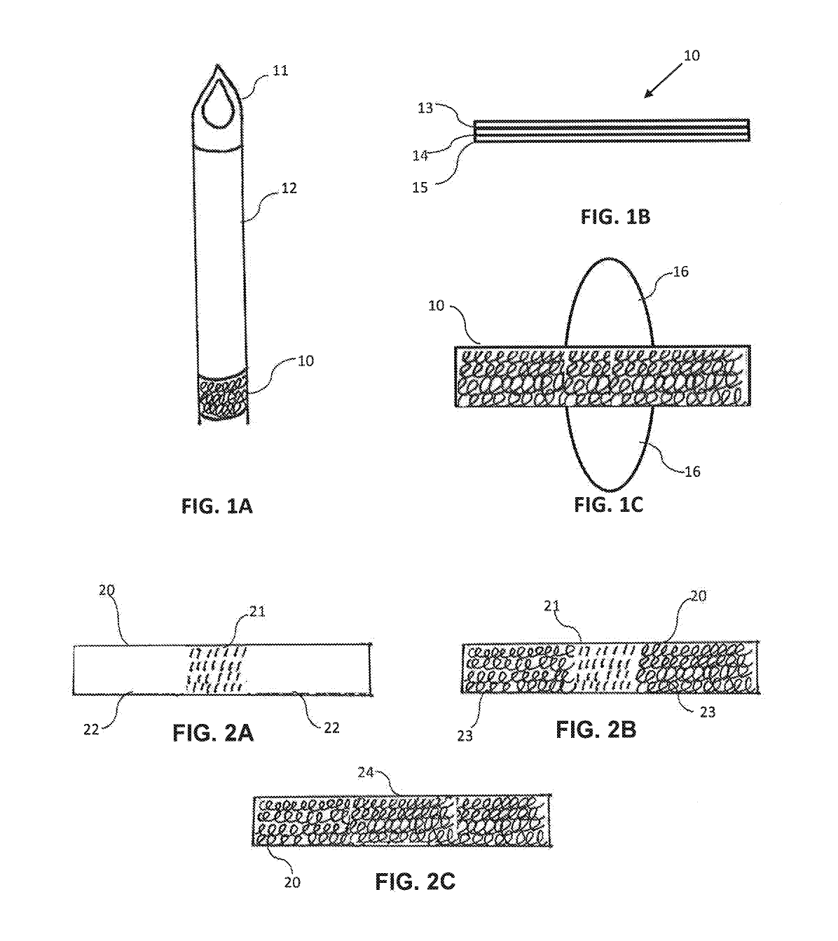

FIG. 1A illustrates the terminal portion of a catheter and includes an insertion device 11, a catheter 12, and a cuff 10. The cuff 10 in one embodiment can be made of absorbent and/or antimicrobial material. The absorbent feature when present can allow the cuff to wick fluids that promote bacterial growth away from the insertion site. The antimicrobial feature can prevent the growth of microorganisms. The above features can decrease infection rate with catheter securement.

The absorbency of the cuff 10 can be provided through inclusion in the cuff 10 of absorbent structures including, without limitation absorbent fibers, particles, or the like. For instance, the cuff 10 can incorporate a hydrophilic material in the form of absorbent fibers in a woven or nonwoven fibrous web. By way of example, an absorbent material of a cuff 10 can include, without limitation, cellulose (e.g., carboxy methyl cellulose), rayon, polysaccharides, alginates, cotton, etc., as well as combinations of absorbent materials.

The cuff 10 can be in the form of a strip or sheet of material that can be attached to a distal portion of a catheter 12 or can be a portion of the catheter 12 as formed. For instance, the cuff 10 can be integrated into the catheter 12 or can be attached to the catheter 12 during manufacture or can be attached at the time of use. In one embodiment, the cuff 10 can be adhered to the catheter by a medical professional once the catheter is placed.

In one embodiment, the cuff 10 can be provided in the form of a strip or sheet as shown in a cross sectional view in FIG. 1B. In this embodiment, the cuff 10 can include a sticky or tacky material for attachment to a catheter 12. For instance, the cuff 10 can include a top layer 13 that upon attachment of the cuff 10 to a catheter 12 can be external to the catheter, i.e., face out from the catheter. In one embodiment, the external or top layer 13 can include an absorbent material such as absorbent fibers, particles, etc. In addition the cuff 10 can include an adhesive layer 14, for instance a pressure sensitive adhesive layer. In addition, the cuff 10 can include a cover layer 15. During use, the cover layer 15 can be removed to display the adhesive layer 14, which can then be utilized to attach the cuff 10 to a catheter 12 as shown in FIG. 1A with the layer 13 on the external surface of the catheter 12, as shown. The external layer 13 can be utilized to attach the catheter to the patient either directly or indirectly, various embodiments of which are detailed herein. The external layer 13 can optionally be absorbent and/or can include one or more antimicrobial agents. For instance, the layer 13 can include one or more absorbent structures, examples of which are described above.

In one embodiment, the cuff 10 may exhibit antimicrobial capability. For instance, the cuff 10 can be formed to include one or more antimicrobial agents as are known in the art. Antimicrobial agents can be provided in any suitable form or fashion including, without limitation, antimicrobial particles or compounds impregnated within the absorbent cuff, coatings on the surface of the absorbent cuff, or combinations thereof. In one embodiment, the cuff can be impregnated with an aqueous solution of an antimicrobial agent, such as a cationic biocide one example of which is an oligo- or polymeric biguanide or salt thereof, for instance polyhexamethylene biguanide (PHMB), chlorhexidine (imidodicarbonimidic diamide), and so forth. Other exemplary antimicrobial agents can include, without limitation, antimicrobial metals, metal salts (e.g. a silver salt) or metal complexes; antimicrobial quaternary ammonium compounds; and benzimidazole and derivatives thereof such as parachlorometaxylenol isothiazolone, benzoisothiazolone and the like.

The cuff design and attachment mechanism can vary. For instance, in those embodiments in which the cuff 10 is directly attachable to the skin of a subject, the cuff can be abraded or roughened to promote better adherence. As illustrated in FIG. 1C, in one embodiment the cuff 10 can have one or more extensions 16 (or ‘wings’) that can extend generally perpendicular to the catheter as illustrated and can be located proximal of the insertion site and essentially flush with the patient's skin during use. The extensions 16 can include an adhesive on the skin contacting side to directly attach the cuff 10 to the skin of a subject.

In one embodiment, the extensions 16 can include an absorbent material and/or an antimicrobial agent. Alternatively, the extensions 16 can be utilized merely for attachment purposes. The extensions 16 can directly attach a cuff 10 to the skin of a patient and can decrease axial rotation and increase overall stability of the catheter 12 to which the cuff 10 is attached.

Referring again to FIG. 1A, a cuff 10 can be attached to a catheter 12 at or relatively close to the insertion site. Any catheter designed for temporary insertion to a subdermal location can be utilized in conjunction with a securement device as disclosed herein including catheters for cardiovascular, urological, gastrointestinal, neurovascular, and ophthalmic applications. A catheter 12 can be formed of any suitable material as is generally known in the art. In general, the catheter 12 can be flexible and can be formed from a polymeric material including any of a range of polymers known in the construction of catheters, including, without limitation, silicone rubber, nylon, polyurethane, polyethylene terephthalate (PET), natural rubbers (latex), and thermoplastic elastomers.

The cuff 10 is generally attached to the catheter 12 at a distance that varies depending on application. In one embodiment, for instance, the cuff 10 can be placed at a site on the catheter 12 that is from 10 to 15 millimeters from the insertion site. In another embodiment, the cuff 10 can be at the insertion side or quite close to the insertion site, for instance about 10 millimeters or less. Of course, the cuff 10 can be at a greater distance from the insertion site as well. The cuff 10 can be of a size so as to extend along an axial length of the catheter 12 as shown in FIG. 1A. For instance, the cuff 10 can extend along an axial length of the catheter 2 inches or less in some embodiments. In addition, while the cuff 10 can completely encircle the catheter 12 in one embodiment, this is not a requirement and in some embodiments, the cuff 10 may wrap only partly around the circumference of a catheter.

As illustrated in FIG. 1, the catheter 12 can be associated with an insertion device 13 such as a trocar or a needle. According to standard practice, the insertion device 13 can be housed within a catheter 12 that can be stabilized on the skin by use of a cuff 10. The insertion device 13 can be provided at the terminal portion of the catheter 12 to puncture the skin and place the catheter terminus within the desired cavity, e.g. an intravenous catheter for use in a vein. During use, once the insertion device 13 and the catheter 12 have been placed subdermally, the insertion device 13 can be removed from the catheter 12 that now has access to the desired cavity. A cuff 10 can be attached to the catheter 12 either prior to or following placement of the catheter terminus in the desired cavity. The cuff 10 can then be directly or indirectly attached to the skin. For instance, the cuff 10 can be directly attached to the skin by use of extensions 16 illustrated in FIG. 1B.

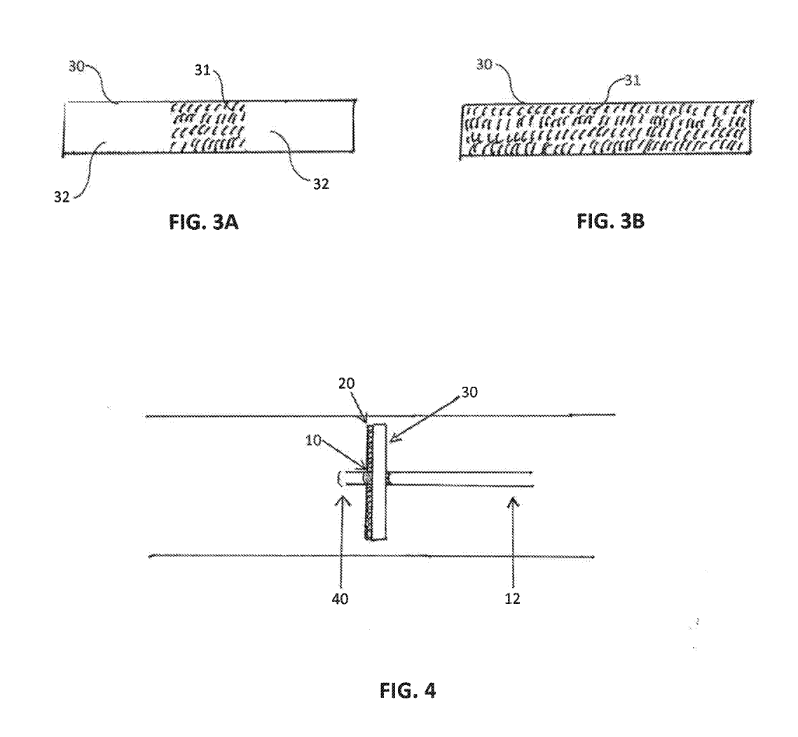

The catheter securement can be adjusted to secure any short or long term catheter that is utilized in any location in the body. As described above, a cuff 10 can be utilized alone to secure a catheter. In other embodiments, a securement device can include one or more additional components to secure a catheter to a patient. For example, in one embodiment, a securement device can include a cover strip 30 as illustrated in FIG. 3A.

The cover strip 30 of FIG. 3A includes a central portion 31 that includes a material that in one embodiment can be attached to a cuff 10. The attachment mechanism between the material of the central portion 31 and the cuff 10 can be of any suitable type. In one embodiment, a hook and loop type of attachment mechanism can be utilized. In this embodiment one of the components (either the cuff or the cover strip) can include the hooks at the surface of the component and the other component can include the loops. For instance, an absorbent material of the cuff 10 can have a pile surface that can function as the loops of a hook and loop type of attachment and the central portion 31 of the cover strip 30 can include a typical hook surface as is generally known in the art at the surface of the central portion 31.

Other fastening mechanisms can alternatively be utilized to attach the cover strip 30 to the cuff 10. For instance, the central portion 31 of the cover strip 30 and the cuff 10 can include magnets that can be utilized to temporarily attach the two together.

In one embodiment, the cover strip 30 can include an absorbent material and/or an antimicrobial agent. The cover strip 30 can include antimicrobial and/or absorbent materials at all or only a portion of the cover strip 30. In addition, when included, the cover strip 30 can include the same or different absorbent materials and/or antimicrobial agents as the material of the cuff 10. For instance, an absorbent material that has optionally been impregnated with an antimicrobial agent and includes a plurality of attachment hooks for a hook and loop attachment type mechanism can be utilized at the surface of the central portion 31.

On either side of the central portion 31 of the cover strip 30 of FIG. 3A the cover strip includes portions 32 that can include an adhesive suitable for attachment to the skin of a patient. The portions 32 of the cover strip 30 can include at the surface any suitable sticky or tacky material (for example a pressure sensitive adhesive) that can be utilized to secure a cuff 10 to a patient's skin. For instance, the portions 32 can include and adhesive layer and cover layer as illustrated in FIG. 1B.

During use, the catheter 12 including the cuff 10 can be located as desired against the patient's skin and the cover strip 30 including portion 31 and portions 32 can be placed by the medical professional over the catheter 12 such that the portions 32 are attached to the patient's skin. In one embodiment, the portion 31 can be attached to the cuff 10, e.g., by the hook and loop fastening mechanism. In another embodiment, the cuff 10 can be held between the cover strip 20 and the patient's skin by friction between the cuff 10 and the portion 31, with no active attachment mechanism between the two. Thus, the catheter 12 can be secured against the patient's skin by use of the cuff 10 and the cover strip 20.

The second or top side of the cover strip (not visible in FIG. 3A) can be any suitable material. For example, the top side of the cover strip can include a nylon or soft plastic material and does not include any adhesive.

In one embodiment, a securement device can include a skin strip 20 that can be utilized in conjunction with a cuff 10 and a cover strip 30. Embodiments of a skin strip 20 are illustrated in FIG. 2A, FIG. 2B, and FIG. 2C. As illustrated in FIG. 2A and FIG. 2B, a skin strip 20 can include a portion 21 that can be adjacent to a cuff 10 when used. In one embodiment, the portion 21 can be attachable to a cuff 10. For example, in one embodiment the portion 21 can include a hook material that can be attachable to a loop material of the cuff 10 (or vice versa) according to a hook and loop attachment mechanism. Of course, other attachment mechanisms such as magnets or adhesive type mechanisms can be used.

On either side of the portion 21 the skin strip 20 can include portions 22 (FIG. 2A), 23 (FIG. 2B) that are configured for removable attachment to a cover strip 30. For instance, in the embodiment of FIG. 2A, the portions 22 can be configured for attachment to the portions 32 of the cover strip of FIG. 3A via an adhesive carried on one or both of the components. For example, both the portions 22 of the skin strip and the portions 32 of the cover strip can include an adhesive. Alternatively, only one of the two strips can carry an adhesive at a surface, and the mating surface can be formed of a material (e.g., a polymer) that can adhere to the adhesive.

Other temporary attachment mechanisms can be utilized between a skin strip and a cover strip. For instance, in one embodiment, a skin strip and a cover strip can be attached to one another by use of a hook and loop attachment mechanism with a length of the catheter sandwiched between the two by use of the cuff 10. For example, and as illustrated in FIG. 2B, a skin strip 20 can include a portion 21 that includes a hook material at a surface that is configured for attachment to the loop material at a surface of a cuff 10. In addition the skin strip 21 includes portions 23 that include a loop material that is configured for attachment to a cover strip 20 that includes a hook material at the aligning portions (e.g, as illustrated in FIG. 3B).

In yet another embodiment illustrated in FIG. 2C, the skin strip 20 can include a material 24 over a surface, for example all hook material or all loop material, and the mating surface of the cuff can include a material that will adhere to the skin strip (e.g., the opposite hook or loop material). In addition, the cover strip 30 can include a material that will adhere to the skin strip (e.g., all hook material or all loop material) on the surface so as to be attached to the skin strip. According to this embodiment, the cuff and the cover strip can both attach to the skin strip and the cuff will remain stabilized between the two strips with the area at which the cuff is adjacent to the cover strip not necessarily including an active attachment mechanism. Of course, the components carrying the hook material and the components carrying the loop material can be altered as desired. For instance, the skin strip can include a loop material, the cover strip can include a hook material, and the cuff can include one or the other to attach to at least one of the cover strip and the skin strip. Thus, in this embodiment, the cuff can be actively attached to one or the other of the cover strip and the skin strip, and the skin strip and the cover strip can be attached to one another at either side of the cuff, thereby securing the cuff between the two and at or near the insertion site of the catheter.

On the opposite side of the skin strip (not shown in FIG. 2A, FIG. 2B, or FIG. 2C) there can be an adhesive layer such as illustrated and discussed above that can be utilized to attach a skin strip to a patient's skin. For instance, the skin strip 20 can include on the second side a layer of a pressure sensitive adhesive and a cover layer overlying the adhesive layer. During use, a medical professional can remove the cover layer to reveal the adhesive layer and place it on the patient's skin. The central portion 21 of the skin strip can then be attached to the cuff 10. Following, a cover strip 30 can over lay both the skin strip 20 and the cuff 10. A portion 31 of the cover strip can be attached to the cuff 10 and portions of the cover strip on either side of this portion (e.g., portions 32 of the cover strip of FIG. 3A or the outer ends of the portion 31 of the cover strip of FIG. 3B) can be attached to portions 22 (FIG. 2A) or portions 23 (FIG. 2B) of the skin strip. Thus, in one embodiment, the skin strip 20 can have a hook portion where it aligns with the cuff 10 and a loop portion elsewhere where it aligns with the cover strip 30 that also includes a hook portion. As with the cover strip and the cuff, a skin strip can optionally include an absorbent material and/or antimicrobial agents at one or more portions to further improve the absorbency and antimicrobial capability of the securement device. The securement unit that includes a skin strip that is temporarily adhered to the skin with a cover strip over laying and attached to the skin strip (and optionally also to the cuff) can thus secure a catheter to a patient without the need for an occlusive adhesive membrane.

FIG. 4 illustrates one embodiment of a method of using a securement device that includes all of a skin strip 20, a cuff 10, and a cover strip 30. As shown, a catheter 12 can be inserted into a body cavity, duct, or vessel. Functionally, it can allow drainage, administration of fluids or gases, access by surgical instruments, and also perform a wide variety of other tasks depending on the type of catheter. Once the catheter 12 is placed into the desired cavity or blood vessel and the insertion device (if any) is withdrawn, then a skin strip 10 can be adhered to the patient's skin just proximal of the puncture site 40 and perpendicular to the longitudinal axis of the catheter 12. In one embodiment and as shown in FIG. 2B a portion 21 of the adhesive skin strip can have a section of hook material that aligns with the puncture site 40 and is located under the catheter 12. A medical professional can push the cuff 10 that has been previously attached to the catheter 12 up against this portion of the skin strip 10 to provide a removable connection between the catheter 12 and the patient. The cover strip 30 can then be placed over the catheter 12 and skin strip 10. The cover strip 30 can be attached to the skin strip 20 at portions on either side of the cuff 10 and optionally also to the cuff 10 at a central portion to provide a strong securement of the catheter 12 to the patient. The material of one or more of the cuff, the skin strip and/or the cover strip can be absorbent and can be impregnated with antimicrobial agents.

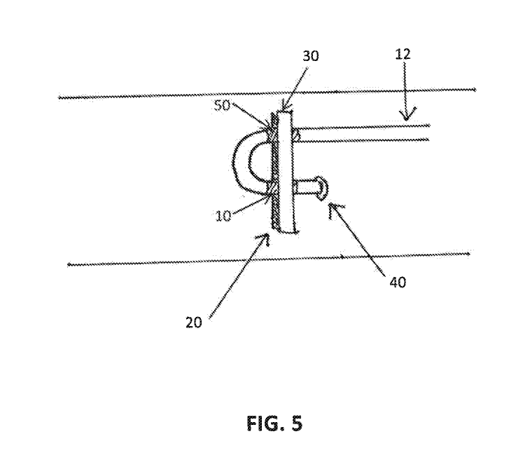

A catheter can be utilized with one or multiple cuffs. FIG. 5 illustrates one embodiment in which a catheter 12 is utilized with a first cuff 10 and a second cuff 50. As illustrated in this embodiment, the cuffs 10, 50 can be secured with a single skin strip 20 and a single cover strip 30 in which the portions for alignment with the cuffs are located according to a predetermined geometric arrangement. Of course, an embodiment can optionally include multiple skin strips and/or multiple cover strips, each of which can be used to secure one or more cuffs. An embodiment including a plurality of cuffs for attachment points to the skin of a patient can be preferred in those embodiments in which it is desirable to attach a catheter (proximal to the insertion site) with a loop, e.g., a 180° turn as illustrated in FIG. 5, for more secure attachment to a patient.

Use of an absorbent and antimicrobial securement device can provide a route for keeping the area near a catheter insertion point dry and free of fluid. No occlusive plastic membrane dressing is required for use in conjunction with a securement device. In one embodiment, a soft and absorbent dressing that is easily changed can be applied in conjunction with the securement device that can allow air circulation and wick away moisture to create an environment that is much less conducive to bacterial infection.

What has been described and illustrated herein is a preferred embodiment of the invention along with some of its variations. The terms, descriptions and figures used herein are set forth by way of illustration only and are not meant as limitations. Those skilled in the art will recognize that many variations are possible within the spirit and scope of the invention in which all terms are meant in their broadest, reasonable sense unless otherwise indicated. Any headings utilized within the description are for convenience only and have no legal or limiting effect.

What is claimed is:

1. A catheter stabilization device comprising a cuff, the cuff being configured to encircle a portion of a catheter and the cuff being configured to be directly or indirectly attachable to skin at an external surface of the cuff.

2. The catheter stabilization device of claim 1, wherein the cuff comprises one or more portions comprising an adhesive.

3. The catheter stabilization device of claim 1, further comprising a cover strip, the cover strip having a length and including a first side and an opposite second side, the cover strip being configured to over lay the cuff upon direct or indirect attachment of the cuff to the skin.

4. The catheter stabilization device of claim 3, the cover strip including an absorbent material and/or an antimicrobial agent.

5. The catheter stabilization device of claim 3, the first side of the cover strip comprising a first portion, a second portion, and a third portion, the second portion being between the first and third portions along a length of the cover strip, the first and third portions comprising an adhesive.

6. The catheter stabilization device of claim 3, wherein the cover strip and the external surface of the cuff include a fastening mechanism providing an attachment between the two.

7. The catheter stabilization device of claim 3, further comprising a skin strip, the skin strip comprising a first side and an opposite second side, the first side including a first portion, a second portion, and a third portion, the second portion being between the first and third portions along a length of the skin strip, the first and third portions being attachable to the first side of the cover strip, the second side of the skin strip including an adhesive for attaching the skin strip to skin.

8. The catheter stabilization device of claim 7, wherein the skin strip and the cover strip include a hook and loop fastening mechanism providing the attachment between the skin strip and the cover strip.

9. The catheter stabilization device of claim 7, the skin strip including an absorbent material and/or an antimicrobial agent.

10. The catheter stabilization device of claim 1, wherein the cuff comprises an absorbent material and/or an antimicrobial agent.

11. The catheter stabilization device of claim 1, the catheter stabilization device including one or more additional cuffs.

12. A method for stabilizing a catheter, the method comprising:
inserting a catheter in a subject;
directly or indirectly attaching a cuff to the subject, the cuff being configured to encircle a portion of the catheter.

13. The method of claim 12, wherein the cuff comprises an antimicrobial agent and/or an absorbent material.

14. The method of claim 12, further comprising overlaying a cover strip over the cuff.

15. The method of claim 14, further comprising attaching the cover strip to the cuff.

16. The method of claim 14, further comprising directly attaching the cover strip to the subject.

17. The method of claim 14, further comprising attaching a first side of a skin strip to a subject and attaching a second side of the skin strip to the cover strip.

18. The method of claim 17, wherein the skin strip and the cover strip are attached to one another by a hook and loop fastening mechanism.

19. The method of claim 17, further comprising attaching the cuff to at least one of the skin strip and the cover strip.

20. The method of claim 12, further comprising directly or indirectly attaching one or more additional cuffs to the subject.
